(19) United States
(12) Patent Application Publication
(10) Pub. No.: US 2009/0229601 A1
(43) Pub. Date: Sep. 17, 2009

(54) Intubating Airway Device

(76) Inventor: Humberto Aguilar, Alvin, TX (US)

Correspondence Address: JAY M. SCHLOFF;INTELLIPEX PLLC, 30200 TELEGRAPH RD, SUITE 245, BINGHAM FARMS, MI, 48025, US

(21) Appl. No.: 12/047,665

(22) Filed: Mar. 13, 2008

(51) Int. Cl. A61M 16/00

(52) U.S. Cl. 128/200.26

(57) ABSTRACT

Disclosed is an intubating airway device for intubating a patient. The intubating airway device comprises a hollow tubular member, a rubber adaptor having an opening, a first connector, an airway tube and a second connector. The hollow tubular member comprises a proximal end, a distal end, and a port that extends outwardly from a peripheral surface of the hollow tubular member. Moreover, the port extends at a portion between the proximal end the distal end. The proximal end is coupled to the rubber adaptor and the distal end is detachably coupled to a first end portion of the first connector. Furthermore, the airway tube is detachably coupled to a second end portion of the first connector. Moreover, the second connector is adapted to be detachably coupled to the first connector, on detachment of the airway tube from the first connector.

### FIELD OF THE INVENTION

The present invention relates to a medical device, and more specifically to an intubating device for supplying respiratory gas to a patient during an intubation process.

### BACKGROUND OF THE INVENTION

During a desaturation condition of a patient, doctors are required to maintain artificial supply of respiratory gas, such as oxygen, to an airway of the patient by an intubation process. More specifically, an endotracheal (ET) tube may be inserted into the windpipe of the patient through the mouth of the patient. Thereafter, the ET tube may be connected to a respiratory gas supply source from one end thereof for supplying the respiratory gas to the windpipe of the patient.

Once the patient returns to a normal breathing condition, the ET tube may be removed from the windpipe of the patient. While removing the ET tube the artificial supply of the respiratory gas may be stopped and it may happen that the patient may be desaturated again due to lack of respiratory gases. Accordingly, the patient may require reintubation after the ET tube has been removed from the patient's windpipe. A delay in the reintubation may result in damage to the brain of the patient.

Moreover, an ET tube is difficult to insert into the windpipe of a patient. Further, the use of ET tube may have to be avoided in certain postures of the patient, for example when a patient is trapped in a sitting position and where tilting of the head of the patient to insert the ET tube is not possible. For such situations, a laryngeal mask airway (LMA) is alternatively utilized for supplying the respiratory gas into the windpipe of the patient.

Accordingly, there persists a need for an intubation device for intubating a patient that avoids the need for reintubating the patient. More specifically, there persists a need of an intubation device that avoids a desaturation of the patient when the ET tube, used for intubation of the patient, is removed. There further persists a need for an intubating device that is versatile to enable utilization of an LMA as well as an ET tube for intubating the patient.

### SUMMARY OF THE INVENTION

In view of the foregoing disadvantages inherent in the prior art, the general purpose of the present invention is to provide an intubating device configured to include all the advantages of the prior art, and to overcome the drawbacks inherent therein.

Accordingly, an object of the present invention is to provide an intubating airway device for intubating a patient that avoids a desaturation of the patient when the intubation is discontinued.

Another object of the present invention is to provide an intubating airway device for intubating a patient that is capable of utilizing an ET tube as well as an LMA for intubating the patient.

In an aspect of the present invention, an intubating airway device is provided. The intubating device comprises a hollow tubular member, a rubber adaptor, a first connector, an airway tube and a second connector. The hollow tubular member comprises a proximal end, a distal end and a port. The port extends outwardly from a peripheral surface of the hollow tubular member. More specifically, the port extends at a portion between the distal end and the proximal end of the hollow tubular member. The rubber adaptor is coupled to the proximal end of the hollow tubular member. Moreover, the rubber adaptor comprises an opening that is adapted to receive an endotracheal (ET) tube, which is connected to an artificial respiration device. The first connector has a tubular structure defining a first end portion and a second end portion. The first end portion of the first connector is detachably coupled to the distal end of the hollow tubular member. The second end portion of the first connector is adapted to be detachably coupled to the airway tube. The airway tube is adapted to be inserted into mouth of a patient. The second connector is adapted to be detachably coupled to the first connector. More specifically, the second connector has a tubular structure defining a first end portion and a second portion, such that the first end portion of the second connector is adapted to be detachably coupled to the second end portion of the first connector on detachment of the airway tube from the second end portion of the first connector.

In another aspect of the present invention, the opening in the rubber adapter of the intubating airway device may be flexible for receiving an ET tube of a predetermined diameter.

In yet another aspect of the present invention, the intubating airway device is adapted to detachably couple a laryngeal mask airway (LMA).

These together with the other aspects of the present invention, along with the various feature of novelty that characterized the present invention, are pointed out with particularity in the claims annexed hereto and form a part of the present invention. For a better understanding of the present invention, its operating advantages, and the specified object attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 illustrate a perspective view of various components of an intubating airway device in a decoupled position, according to an embodiment of the present invention;

FIG. 2 illustrates a first configuration of the intubating airway device of FIG. 1 utilizing an ET tube for intubating a patient, according to an embodiment of the present invention; and

FIG. 3 illustrates a second configuration of the intubating airway device of FIG. 1 utilizing an LMA for intubating a patient, according to another embodiment of the present invention.

Like reference numerals refer to like parts throughout the description of several views of the drawings.

### DETAILED DESCRIPTION OF THE INVENTION

For a thorough understanding of the present invention, reference is to be made to the following detailed description, including the appended claims, in connection with the above-described drawings. Although the present invention is described in connection with exemplary embodiments, the present invention is not intended to be limited to the specific forms set forth herein. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The term “first”, “second”, “distal”, “proximal” and the like, herein do not denote any order, quantity or importance, but rather are used to distinguish one element from another, and the terms “a” and “an” herein do not denote a limitation of quantity, but rather denote the presence of at least one of the referenced item.

The present invention provides an intubating airway device for intubating a patient. More specifically, the intubating airway device is capable of avoiding a desaturation of the patient. The intubating airway device is capable of incorporating different types of intubation tubes, such as an ET tube and a LMA, for supplying respiratory gas and medicines to the patient.

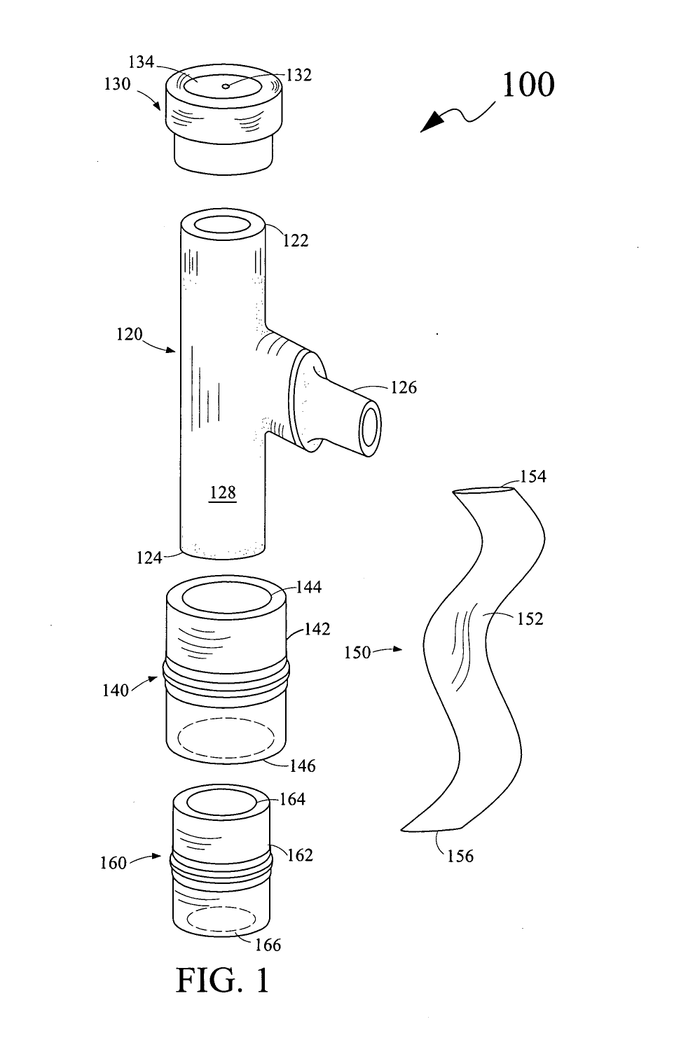

Referring to FIG. 1, the intubating airway device, such as an intubating airway device 100 (hereinafter referred to as ‘airway device 100’), for intubating the patient, is shown. More specifically, FIG. 1 illustrates a perspective view of various components of the airway device 100 in a decoupled position, according to an exemplary embodiment of the present invention.

The airway device 100 includes a hollow tubular member 120 (hereinafter referred to as ‘tubular member 120’), a rubber adaptor 130, a first connector 140, an airway tube 150 and a second connector 160. The tubular member 120 includes a proximal end 122 and a distal end 124. The proximal end 122 and the distal end 124 have substantially equal diameters. Moreover, the tubular member 120 includes a port 126 extending outwardly from a peripheral surface 128 of the tubular member 120 and lying at a portion between the proximal end 122 and the distal end 124. In an embodiment of the present invention, the port 126 may extend transversally from the peripheral surface 128 of the tubular member 120. More specifically, the port 126 and the tubular member 120 may configure a T-shaped structure. However, it will be obvious to a person skilled in the art that the present invention is not limited to structure as described herein and the port 126 may extend at any inclination from the peripheral surface 128. Moreover, the port 126 is adapted to be detachably coupled to an artificial respiration device, such as an anesthesia circuit, for receiving respiratory gas, such as oxygen.

Moreover, to the proximal end 122 of the tubular member 120, the rubber adaptor 130 may be coupled. The rubber adaptor 130 includes an opening 132 configured on a front face 134 of the rubber adaptor 130. The opening 132 is adapted to receive therein an ET tube (not shown) for intubating the patient. In an embodiment of the present invention, the opening 132 may be flexible, such that the opening 132 is capable of receiving the ET tube, of predetermined diameters. For example, the opening 132 may be adapted to receive ET tubes with diameter ranging between about 3 to about 10.5 mm. Moreover, the rubber adaptor 130 may be made of non-latex rubber. The rubber adaptor 130 enables the ET tube received within the opening 132 to be directed towards the distal end 124 of the tubular member 120.

The distal end 124 of the tubular member 120 is adapted to detachably couple the first connector 140 thereto. The first connector 140 has a tubular structure 142 defining a first end portion 144 and a second end portion 146. The first end portion 144 and the second end portion 146 may have substantially equal diameters. Moreover, the diameter of the first end portion 144 is comparatively greater than the diameter of the distal end 124 of the tubular member 120, such that the first end portion 144 is received on the distal end 124 of the tubular member 120. Accordingly, the first connector 140 is coupled to the tubular member 120.

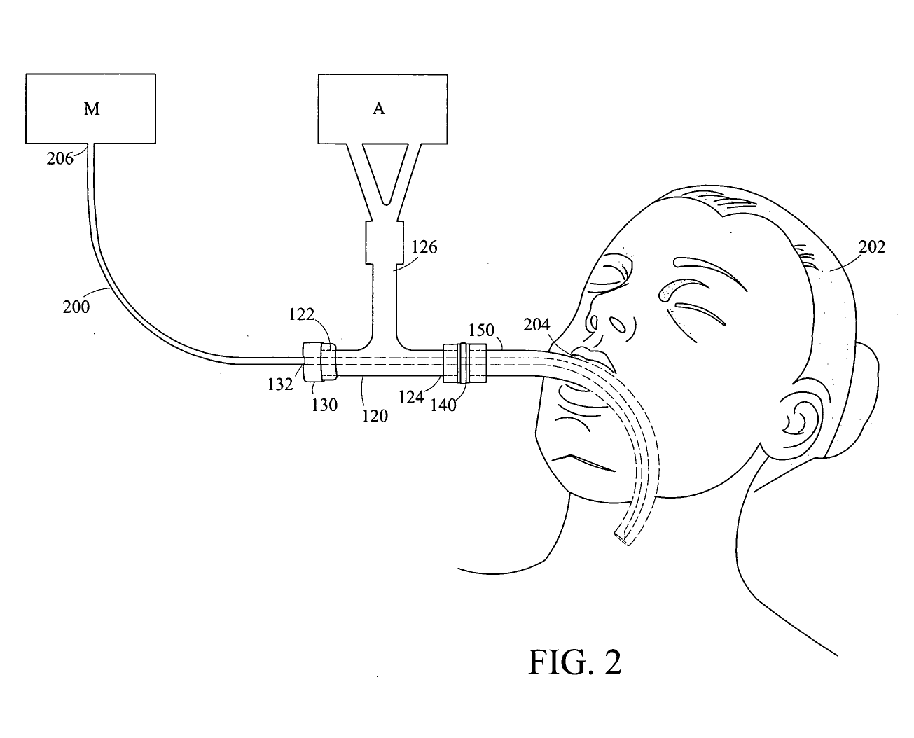

The first connector 140 is further adapted to couple the airway tube 150 to the tubular member 120, in accordance with an embodiment of the present invention, which will be further explained in conjunction with FIG. 2. The airway tube 150 includes end portions 152 and 154. The airway tube 150 is adapted to be inserted through mouth of the patient for enabling intubation thereof. More particularly, the end portion 152 of the airway tube 150 is detachably coupled to the second end portion 146 of the first connector 140. The end portion 154 of the airway tube 150 is adapted to be inserted through the mouth of the patient. Moreover, a diameter of the airway tube 150 is substantially similar to the diameter of the proximal end 122 and the distal end 124 of the tubular member 120.

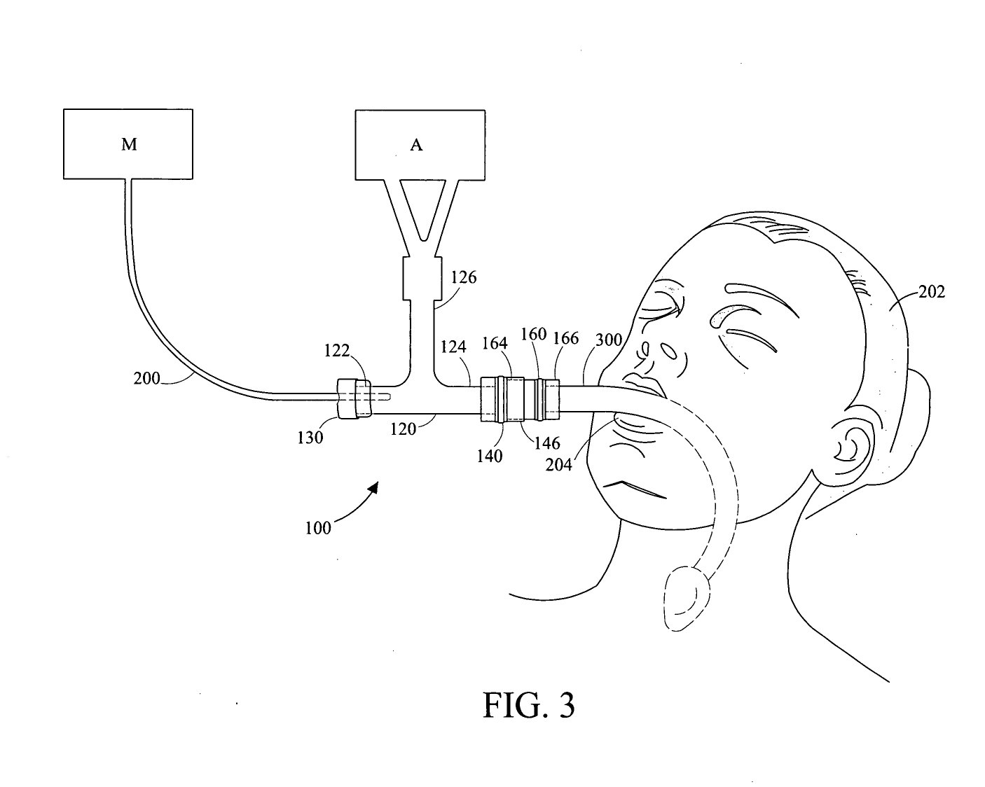

Further, the airway tube 150 may be detached from the first connector 140 to detachably couple the second connector 160 thereto, in accordance with another embodiment of the present invention, which will be further explained in conjunction with FIG. 3. The second connector 160 has a tubular structure 162 defining a first end portion 164 and a second end portion 166. The first end portion 164 of the second connector 160 is adapted to be detachably coupled to the second end portion 146 of first connector 140, when the airway tube 150 has been detached from the first connector 140. The second end portion 166 of the second connector 160 may be detachably coupled to an LMA. Moreover, the second connector 160 is similar in structural configuration to the first connector 140 except that the second connector 160 is comparatively smaller in size than the first connector 140.

In view of the foregoing, the various components of the intubating airway 100, more specifically the tubular member 120, rubber adaptor 130, the first connector 140, the airway tube 150 and the second connector 160, may be selectively connected to configure different configurations of the airway device 100. For example, in an embodiment of the present invention, the tubular member 120, the rubber adaptor 130, the first connector 140, and the airway tube 150 may be coupled to configure a “first configuration” of the airway device 100. The “first configuration” will be further explained in conjunction with FIG. 2. In another embodiment of the present invention, the tubular member 120, the rubber adaptor 130, the first connector 140, and the second connector 160 may be coupled to configure a “second configuration” of the intubating airway device 100. The “second configuration” will be further explained in conjunction with FIG. 3. Moreover, the tubular member 120, the first connector 140, the airway tube 150 and the second connector 160 of the airway device 100 are preferably made of plastic material. However, it will be obvious to the person skilled in the art that any other material providing sufficient structural integrity to the tubular member 120, the first connector 140, the airway tube 150 and the second connector 160 may also be utilized.

FIG. 2 illustrates the first configuration of the airway device 100 utilizing an ET tube 200 for intubating a patient 202, according to an embodiment of the present invention. The intubating device, as described herein, would hereinafter be referred to as “ET airway device”. The ET airway device may be configured by coupling the rubber adaptor 130 on the proximal end 122 of the tubular member 120, and coupling the first connector 140 to the distal end 124 of the tubular member 120. Moreover, the airway tube 150 is coupled to the first connector 140 and is inserted into a windpipe (not shown) of the patient 202 through his/her mouth 204. The opening 132 of the rubber adaptor 130 is adapted to receive the ET tube 200. More specifically, the ET tube 200 passes through the tubular member 120 and the first connector 140 into the airway tube 150, as shown in FIG. 2. As explained in conjunction with FIG. 1, the diameter of the airway tube 150 is substantially similar to the tubular member 120, thereby enabling the ET tube 200 to easily pass through the tubular member 120 into the airway tube 150. Moreover, an end portion 206 of the ET tube 200 may be connected to an artificial respiration device, such as a medical ventilator ‘M’, for receiving respiratory gas therefrom.

Moreover, the port 126 is detachably coupled to an artificial respiration device, such as an anesthesia circuit ‘A’. The anesthesia circuit ‘A’ may be operated by a user to supply respiratory gas to the ET airway device when desired.

In use, the ET tube 200 configures a path between the ventilator device ‘M’ and the airway tube 150 for supplying the respiratory gas to the windpipe of the patient 202 through the airway tube 150. Accordingly, the ET airway device may be utilized for intubating the patient 202 while he/she is experiencing a desaturation condition.

Once the patient 202 recovers from the desaturation condition, the ET tube 200 may be retrieved from the airway tube 150 and the medical ventilator ‘M’ may be disabled to stop the flow of the respiratory gas therefrom. However, during the removal of the ET tube 200, the user may enable the anesthesia circuit ‘A’. The respiratory gas supplied by the anesthesia circuit ‘A’ enters the tubular member 120 from the port 126. Thereafter, the respiratory gas passes into the airway tube 150 for supplying the respiratory gas to the windpipe of the patient. Accordingly, a continuous supply of the respiratory gas is maintained in the windpipe of the patient 202 while the ET tube 200 is being removed, thereby avoiding a desaturation of the patient 202. The ET airway device thereby avoids a need for reintubating the patient 202 after the ET tube 200 has been removed from his/her windpipe.

While using the first configuration, due to a certain posture of the patient 202, it may happen that the ET tube 200 may not be easily inserted into the windpipe of the patient 202. On encountering such a situation, an LMA may be utilized for intubating the patient 202. Accordingly, the ET tube 200 may be retrieved from the airway tube 150 and the airway tube 150 may be detached from the first connector 140. Thereafter, the second connector 160 having an LMA coupled thereto may be connected to the first connector 140 to configure the second configuration of the airway device 100 for intubating the patient.

FIG. 3 illustrates the second configuration of the airway device 100 utilizing an LMA 300 for intubating the patient 202, according to another embodiment of the present invention. The intubating device utilizing the LMA 300 may be hereinafter referred to as “LMA airway device”. The LMA airway device may be configured by coupling the rubber adaptor 130 on the proximal end 122 of the tubular member 120 and coupling the first connector 140 to the distal end 124 of the tubular member 120. Moreover, the LMA airway device includes the second connector 160 coupled to the first connector 140. More specifically, the first end portion 164 of the second connector 160 is detachably coupled to the second end portion 146 of first connector 140. The LMA 300 may be coupled to the second end portion 166 of the second connector 160. Moreover, the port 126 of the tubular member 120 is connected to the anesthesia circuit ‘A’, which supplies respiratory gas to the LMA airway device.

In use, the LMA 300 may be inserted through the mouth 204 of the patient 202 into the windpipe (not shown) of the patient 202. The anesthesia circuit ‘A’ is operated to supply the respiratory gas that passes through the tubular member 120, the first connector 140 and the second connector 160 into the LMA 300. Accordingly, the LMA airway device may be utilized for supplying respiratory gas to the patient 202.

The intubating airway device, as described herein, may be advantageously used for intubating a patient, such that a desaturation condition of the patient is avoided. Further, the intubating airway device is capable of incorporating ET tubes of different diameters to supply different amounts of respiratory gas according to requirement of the patient. Furthermore, the intubating airway device enables use of an LMA for intubating the patient when the ET tube can not be used due to a critical condition of the patient.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present invention.

What is claimed is:

1. An intubating airway device, comprising:
a hollow tubular member comprising a proximal end, a distal end, and a port extending outwardly from a peripheral surface of the hollow tubular member, the port extending at a portion between the proximal end the distal end;
a rubber adaptor adapted to be coupled to the proximal end of the hollow tubular member, the rubber adaptor having an opening adapted to receive an endotracheal tube connected to an artificial respiration device;
a first connector having a tubular structure defining a first end portion and a second end portion, wherein the first end portion of the first connector is adapted to be detachably coupled to the distal end of the hollow tubular member;
an airway tube adapted to be detachably coupled to the second end portion of the first connector, wherein the airway tube is adapted to be inserted through mouth of a patient; and
a second connector having a tubular structure defining a first end portion and a second end portion, wherein the first end portion of the second connector is adapted to be detachably coupled to the second end portion of the first connector on detachment of the airway tube from the second end portion of the first connector.

2. The intubating airway device of claim 1, wherein the second end portion of the second connector is adapted to be detachably coupled to a laryngeal mask airway.

3. The intubating airway device of claim 1, wherein the opening of the rubber adaptor is flexible to receive an endotracheal tube of a predetermined diameter.

4. The intubating airway device of claim 1, wherein the port is adapted to be detachably coupled to an anesthesia circuit.

5. The intubating airway device of claim 1, wherein a diameter of the airway tube and a diameter of the distal end of the hollow tubular member is substantially equal.

6. The intubating airway device of claim 1, wherein the hollow tubular member is configured to form a T-shaped structure.

7. The intubating airway device of claim 1, wherein a material of the hollow tubular member is plastic.

8. The intubating airway device of claim 1, wherein a material of the first connector is plastic.

9. The intubating airway device of claim 1, wherein a material of the second connector is plastic.

10. The intubating airway device of claim 1, wherein a material of the airway tube is plastic.

11. The intubating airway device of claim 1, wherein the rubber adaptor is made of non-latex rubber.
